(19) United States
(12) Patent Application Publication
(10) Pub. No.: US 2014/0228314 A1
(43) Pub. Date: Aug. 14, 2014

(54) METHOD FOR PREPARATION OF CHITOSAN OLiGOSACCHARIDES COMPOSITION AND MIXTURE OF CHITOSAN OLiGOSACCHARIDES PREPARED THEREFROM

(71) Applicant: KUNPOONG BIO CO., LTD, Seoul (KR)

(72) Inventors: Chen-Gum OH, Seoul (KR); Young-In KWON, Daejeon (KR); Jong-Wook LEE, Jeju-si (KR); Seong-Chul KIM, Jeju-si (KR)

(73) Assignee: KUNPOONG BIO CO., LTD, Seoul (KR)

(21) Appl. No.: 14/079,706

(22) Filed: Nov. 14, 2013

(30) Foreign Application Priority Data
Feb. 12, 2013 (KR) 10-2013-0014961

(51) Int. Cl. C12P 19/26; A23L 2/52; A61K 31/7016

(52) U.S. Cl. CPC C12P 19/26; A61K 31/7016; A23L 2/52
USPC 514/53; 435/99

(57) ABSTRACT

Disclosed is a method for preparation of a chitosan oligosaccharide composition capable of increasing a content of disaccharide in chitosan oligosaccharide with excellent effects of reducing HbAlc and blood glucose as well as excellent thermal stability. The method for preparation of a chitosan oligosaccharide composition includes: adding chitosan to distilled water to swell the chitosan; firstly treating the chitosan with a chitosanase to prevent the chitosan solution from becoming a state of being viscose, then, adding an organic acid to a mixed solution of the chitosan and chitosanase at a predetermined time interval bit by bit over several additions; allowing mutual reaction between the chitosan, chitosanase and organic acid; and inactivating the chitosanase to obtain the chitosan oligosaccharide composition. Further, a medicine and/or health supplement food which is effective in preventing and treating diabetes, including at least 40% of disaccharide without monosaccharide, is also disclosed.

### BACKGROUND OF THE INVENTION

1. Field of the Invention

The present invention relates to a novel method for preparation of a chitosan oligosaccharide composition and a mixture of chitosan oligosaccharides prepared using the same, and more particularly, to a method for preparation of a chitosan oligosaccharide composition with advantages in that it may prevent a chitosan solution from becoming a state of being viscous, increase the content of disaccharide in chitosan oligosaccharide with excellent effects in terms of reducing glycosylated hemoglobin and blood glucose, and have excellent thermal stability, as well as a mixture of the chitosan oligosaccharides prepared by the foregoing method.

In addition, the present invention relates to a novel chitosan oligosaccharide mixture with superior effects in terms of reducing blood glucose, as compared to existing chitosan oligosaccharide formulations having anti-diabetic effects reported in the related art, wherein the foregoing effects are demonstrated through animal experiments and clinical trials, and a decrease in glycosylated hemoglobin (HbA1c) as a new diagnosis item and a standard for assessment of a long term blood glucose control, thereby preventing diabetes complications.

Further, the present invention relates to a mixture of a chitosan oligosaccharide ascorbic acid salt and a chitosan oligosaccharide succinic acid salt with superior effects in terms of reducing HbAlc and blood glucose, as compared to separately administered chitosan oligosaccharide ascorbic acid salt and/or separately administered chitosan oligosaccharide succinic acid salt.

Further, the present invention relates to a medicine and/or health supplement food which is effective in preventing and treating diabetes, including chitosan oligosaccharide that contains at least 40 wt. % of disaccharide without monosaccharide and has superior effects in terms of reducing HbAlc and blood glucose in Type II diabetes as well as excellent thermal stability.

2. Description of the Related Art

Chitosan is obtained by grinding mushrooms, carapaces of crustaceans (crabs, prawns, etc.) or bones of mollusks (squids, cuttlefishes, etc.), deproteinizing and desalting chitin, and deacetylating the same. In view of chemistry, a material including 70% or more of D-glucosamine produced by adding an alkaline component to chitin, as a polymeric polysaccharide formed by β-(1,4) polymerization bonding of N-acetyl-D-glucosamine monomers, to remove an acetyl group from N-acetyl-D-glucosamine, is referred to as ‘chitosan’ (Korea Ministry of Food and Drug Safety, Korean Food Additives Codex). Further, chitosan oligosaccharide is an oligosaccharide (having 2 to 50 glucoses) obtained by severing the chitosan, as a polysaccharide polymer, through chemical degradation using an inorganic acid or enzymatic degradation, and a degree of polymerization is determined depending upon the number of polymerized glucoses. For instance, when the chitosan oligosaccharide is a disaccharide having 2 glucoses, a degree of polymerization is 2. Likewise, a chitosan oligosaccharide containing 15 glucoses will have a degree of polymerization of 15.

It has been reported that a polymer chitosan is a polysaccharide not digested by animal digestive enzymes, and has characteristics of animal dietary fibers, thus being partially degraded by lysozyme, chitinase and/or chitosanase which are secreted by intestinal bacteria, and a very tiny quantity thereof is absorbed in the body of the animal. A major function of the polymer chitosan is to promote excretion of bile acid through binding of the polymer chitosan to the bile acid in the small intestine, which in turn, leads to an increase in consumption of cholesterol as a precursor of the bile acid. Therefore, the polymer chitosan is known to have the effect of decreasing a content of cholesterol in the body. In addition, chitosan is a cationic polymeric material and reported to have anti-cancer, anti-microbial and immunopotential effects through a variety of animal experiments, therefore, various attempts to use chitosan in medical applications have proceeded in recent years.

Dislike chitin or chitosan, since chitosan oligosaccharide has relatively favorable water-solubility and excellent absorption within the body and various physiological functions thereof have been found recently, it has drawn attention as a novel functional material. According to in vitro researches, antimicrobial, antifungal, antioxidant and anti-mutational functions of chitosan have been reported. Further, in animal experiments, chitosan was known to have physiologically active functions.

With regard to chitosan oligosaccharide and its efficacy, a patent published and owned by the present applicant before filing the present application, that is, Korean Patent Registration No. 10-0506710 discloses a chitosan oligosaccharide composition including tetra- and penta-saccharides as major components and its effects of reducing blood glucose. Also, U.S. Pat. No. 5,981,510 describes anti-diabetic effects attained using chitosan oligosaccharide of acetate salt. However, a method for preparation of the chitosan oligosaccharide composition described in the foregoing Korean Patent Registration No. 10-0506710 involves a limitation in manufacturing a chitosan oligosaccharide composition having less content of disaccharide in chitosan oligosaccharide, which is known to have superior blood glucose reduction effects and anti-diabetic effects. On the other hand, it was found that chitosan oligosaccharide of the acetate salt used in U.S. Pat. No. 5,981,510 entails a problem of thermal stability.

Diabetes is a disease that progresses to complications such as loss of sight, heart disease, stroke, heart failure, or the like, which in turn, leads to patient death. Although many diabetes patients perform self-monitoring of blood glucose and undergo blood glucose control, the disease may still progress to the point of complications, leading to death. Reasons for difficulties in preventing diabetes complications include: still not correctly understanding the causes for the occurrence of diabetes; as well as genetic factors, environmental factors such as stress, obesity, aging, drinking alcohol, etc., and social causes based on a drastic change in dietary habits, which have been revealed to be a combination of these factors. Accordingly, it is difficult to manage, control and/or prevent various diabetes complications, and correctly and precisely determine whether the likelihood of diabetes complications has increased or reduced and whether blood glucose control is proceeding well, by the monitoring of only blood glucose.

Since a general blood glucose test requires an empty stomach (fasting plasma glucose: FPG), and blood glucose levels are significantly varied by meals, exercise and administration of a diabetes drug, or the like, and a blood glucose level is indicated only at a specific moment, it is difficult to accurately determine the true condition in relation to long term blood glucose control.

In other words, although blood glucose is used as a diagnosis standard for diabetes, it is still disputed as to which one should be selected among values of, on an empty stomach, after meals, and in 2 hours after a glycemic load test, as the diagnosis standard for diagnosing diabetes and/or impaired glucose tolerance. Further, there is a large difference in blood glucose levels depending upon types of specimens for measurement of blood glucose and diagnosis of diabetes. In addition, blood glucose levels are different according to types of blood in blood-gathering, that is, venous blood, artery blood or capillary blood, and also vary according to fasting or not, or an intake time. For instance, when fasting in the early morning, a difference in blood glucose concentration between an artery and a vein may be around 10 mg/dL. On the other hand, such a difference after a meal may range from 20 to 50 mg/dL. Therefore, blood glucose control using blood glucose causes problems.

In consideration of the foregoing problems, the importance of glycosylated hemoglobin (HbAlc) in blood used for diabetes diagnosis as well as blood glucose level has gradually increased in recent years. HbAlc is formed by bonding glucose in the blood to hemoglobin (Hb) of a red blood cell transporting oxygen. Since a glucose fraction bonded to the red blood cell survives for about 120 days which is a life-span of a red blood cell, an average blood glucose concentration over 2 to 3 months of a diabetes patient can be found by testing the glucose fraction. Accordingly, it is possible to correctly monitor and determine overall conditions of blood glucose control. Further, an HbAlc value is associated with diabetes complications such as (diabetic) retinopathy, (diabetic) nephropathy, or the like. It has been disclosed that, if an HbAlc value is decreased by 1%, cardiac infarction is reduced by 14%, cataracts are reduced by 19%, microangiopathy is reduced by 37%, peripheral vessel diseases are reduced by 43%, and mortality caused by diabetes is reduced by 21%. Elizabeth Selvin in the United States undertook a study relating to the suitability of standards for diabetes diagnosis and for use of HbAlc as an indicator and analyzed the correlation between the relevant indicators and diabetes complications from patients who have participated in various clinical trials for 15 years. In this regard, it was demonstrated that, when an HbAlc value is 6.5% or more, a risk for the occurrence of diabetes is 16.47-fold higher, as compared to an HbAlc value of 5.0 to 5.5%. Further, it was reported that a possibility of the occurrence of coronary artery diseases such as a heart attack is 1.95-fold higher while a stroke risk is 3.16-fold higher. As such, blood glucose only indicates a glucose value at one moment while HbAlc can be used as an indicator for diabetes diagnosis and for evaluating prevention and reduction of diabetes complications. Results of research have demonstrated that a 1% decrease in HbAlc significantly reduces a risk for the occurrence of diabetes complications. HbAlc is indicated in a unit of percentage (%) while blood glucose is indicated in mg/mL. The United States Diabetes Society converted the unit of % for HbAlc into a blood concentration to indicate HbAlc, adopted it as a standard, and recommended an HbAlc test as a new basis for diabetes diagnosis and management thereof (see Table 1 in Experimental Example 4 below).

Under the foregoing circumstances, as a result of intensive and repeated studies to solve problems of conventional technologies in chitosan oligosaccharide applications, as described above, and to develop a novel chitosan oligosaccharide composition, the present inventors have found that, if a chitosan solution is firstly treated using an enzyme and an organic acid is slowly added to the chitosan solution bit by bit over several additions, a problem of altering the chitosan solution in a viscous state by firstly adding the organic acid to the solution, can be overcome, therefore, a content of disaccharide in chitosan oligosaccharide with excellent effects in terms of reducing HbAlc and blood glucose is increased, thereby completing the present invention.

Further, the present inventors have found that a mixture of chitosan oligosaccharide ascorbic acid salt and chitosan oligosaccharide succinic acid salt has superior effects in terms of reducing HbAlc and blood glucose, as compared to separately administering chitosan oligosaccharide ascorbic acid salt and/or separately administered chitosan oligosaccharide succinic acid salt, thereby completing the present invention.

### SUMMARY OF THE INVENTION

In order to overcome the foregoing problems, an object of the present invention is to provide a method for preparation of a chitosan oligosaccharide composition, including: firstly treating a chitosan solution with an enzyme; and then, slowly adding an organic acid to the chitosan solution bit by bit over several additions, so that the composition may prevent a chitosan solution from becoming a state of being viscous, increase a content of disaccharide in chitosan oligosaccharide with excellent effects in terms of reducing glycosylated hemoglobin and blood glucose, and have excellent thermal stability, as well as a mixture of the chitosan oligosaccharides prepared by the foregoing method.

In addition, another object of the present invention is to provide a novel chitosan oligosaccharide mixture with superior effects in terms of reducing blood glucose, as compared to existing chitosan oligosaccharide formulations having anti-diabetic effects reported in the related art, wherein the foregoing effects are demonstrated through animal experiments and clinical trials, and a decrease in glycosylated hemoglobin (HbAlc) as a new diagnosis item and a standard for assessment of a long term blood glucose control, thereby preventing diabetes complications.

Further, another object of the present invention is to provide a mixture of a chitosan oligosaccharide ascorbic acid salt and a chitosan oligosaccharide succinic acid salt with superior effects in terms of reducing HbAlc and blood glucose, as compared to separately administered chitosan oligosaccharide ascorbic acid salt and/or separately administered chitosan oligosaccharide succinic acid salt.

Further, another object of the present invention is to provide a medicine and/or health supplement food which is effective in preventing and treating diabetes, including chitosan oligosaccharide that contains at least 40 wt. % of disaccharide without monosaccharide and has superior effects in terms of reducing HbAlc and blood glucose as well as having excellent thermal stability.

In order to accomplish the foregoing objects, the present invention provides a method for preparation of a chitosan oligosaccharide composition that can increase a content of disaccharide in chitosan oligosaccharide with excellent effects in terms of reducing HbAlc and blood glucose and has excellent thermal stability.

Further, the present invention provides a medicine and/or health supplement food which is effective in preventing or treating diabetes with excellent effects in terms of reducing HbAlc and blood glucose, including at least 40 wt. % of disaccharide without monosaccharide.

A degree of polymerization of the chitosan oligosaccharide used in the present disclosure may be defined as an ‘n’ value of a compound represented by Formula I below:

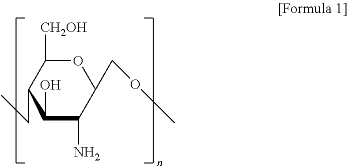

For instance, chitosan oligosaccharide having disaccharide may have an ‘n’ value of 2 in Formula 1, chitosan oligosaccharide having trisaccharide may have an ‘n’ value of 3 in Formula 1, and chitosan oligosaccharide having tetrasaccharide may have an ‘n’ value of 4 in Formula 1.

Hereinafter, the present invention will be described in more detail.

A method for preparation of a chitosan oligosaccharide composition of the present invention includes: adding chitosan to distilled water to swell the chitosan; firstly treating the chitosan with a chitosanase to prevent the chitosan solution from becoming a state of being viscous, then, adding an organic acid to a mixed solution of the chitosan and chitosanase at a predetermined time interval bit by bit over several additions; allowing mutual reaction between the chitosan, chitosanase and organic acid; and inactivating the chitosanase to obtain the chitosan oligosaccharide composition.

According to one embodiment of the present invention, a method for preparation of a chitosan oligosaccharide composition may further include drying the obtained chitosan oligosaccharide composition to be in a powder form.

According to another embodiment of the present invention, a method for preparation of a chitosan oligosaccharide composition may include drying the obtained chitosan oligosaccharide composition to be in a powder form, by at least one drying process selected from a group consisting of natural drying, freeze-drying and hot air drying.

With regard to the method for preparation of a chitosan oligosaccharide composition according to one embodiment of the present invention, the obtained chitosan oligosaccharide composition may contain at least 40 wt. % of disaccharide in the chitosan oligosaccharide to 100 wt. % of a total composition without monosaccharide.

With regard to the method for preparation of a chitosan oligosaccharide composition according to one embodiment of the present invention, the obtained chitosan oligosaccharide composition may have the effects of decreasing an HbAlc value and blood glucose.

With regard to the method for preparation of a chitosan oligosaccharide composition according to one embodiment of the present invention, an organic acid may be added to a mixed solution of chitosan and chitosanase at an interval of 10 minutes bit by bit over six additions.

With regard to the method for preparation of a chitosan oligosaccharide composition according to one embodiment of the present invention, the organic acid may be at least one selected from a group consisting of ascorbic acid and succinic acid.

With regard to the method for preparation of a chitosan oligosaccharide composition according to one embodiment of the present invention, the obtained chitosan oligosaccharide may be a chitosan oligosaccharide ascorbic acid salt, chitosan oligosaccharide succinic acid salt or a mixture thereof.

The chitosan oligosaccharide mixture of the present invention is preferably a mixture of the chitosan oligosaccharide ascorbic acid salt and chitosan oligosaccharide succinic acid salt and, more preferably, a content of the chitosan oligosaccharide succinic acid salt ranges from 5 to 35 wt. % to 100 wt. % of a total mixture. With regard to the chitosan oligosaccharide mixture according to one embodiment of the present invention, the chitosan oligosaccharide ascorbic acid salt and chitosan oligosaccharide succinic acid salt may be present in a relative ratio by weight of 7:3.

A composition including the chitosan oligosaccharide mixture of the present invention may reduce HbAlc to thus prevent and treat metabolic diseases and diabetes, be applicable in medicines and/or health supplement foods for treatment and prevention of diabetes complications and, in particular, be easily applied to foods. Types of the food in the present invention are not particularly limited, however, may include drinks, beverages, or the like. Further, the chitosan oligosaccharide mixture of the present invention may also be used in tea bags, capsules, powder, or tablet products.

In such a food as described above, a content of the chitosan oligosaccharide mixture of the present invention is not particularly limited. However, for general food products, the chitosan oligosaccharide mixture of the present invention may be included in an amount of about 0.01 to 50 wt. % to a total weight of a final food product. On the other hand, for health supplement foods, the chitosan oligosaccharide mixture of the present invention may be included in an amount of about 0.01 to 100 wt. % to a total weight of a final food product. In particular, with regard to a composition including the chitosan oligosaccharide mixture of the present invention in order to achieve the effects of reducing HbAlc and blood glucose, it may be formulated at an administration dose of 300 to 3,000 mg daily in terms of content of oligoglucosamine.

Meanwhile, a pharmaceutical composition including the chitosan oligosaccharide mixture of the present invention may be formulated in various forms for oral administration. Such a formulation for oral administration may include, for example, tablets, pills, hard/soft capsules, liquids, suspensions, emulsions, syrups, granules, elixirs, or the like. These formulations may contain diluents (i.e., lactose, dextrose, sucrose, mannitol, sorbitol, cellulose and/or glycine), lubricants (i.e., silica, talc, stearic acid and magnesium or calcium salts thereof, and/or polyethyleneglycol), other than oligoglucosamine as an active ingredient. The tablet may further contain a binder such as magnesium aluminum silicate, starch paste, gelatin, tragacanth, methyl cellulose, sodium carboxymethyl cellulose and/or polyvinyl pyrrolidine. Optionally, a disintegrant such as starch, agar, alginic acid or sodium salts thereof, or a boiling mixture and/or absorbent, a coloring agent, flavor and sweetener may be further included.

Further, the pharmaceutical composition including the chitosan oligosaccharide mixture of the present invention may be sterilized and/or contain supplements (or additives), for example, a preservative, stabilizer, hydrating agent or emulsion enhancer, salts for osmosis control, and/or buffer, and other therapeutically useful materials. Moreover, the pharmaceutical composition may be manufactured into formulations according to any conventional method such as mixing, granulating or coating.

The present invention may also provide a method for preparation of a chitosan oligosaccharide composition, including: firstly treating a chitosan solution with an enzyme; and then, slowly adding an organic acid to the chitosan solution bit by bit over several additions, so that the composition may prevent the chitosan solution from becoming a state of being viscous, increase a content of disaccharide in chitosan oligosaccharide with excellent effects in terms of reducing HbAlc and blood glucose, and have excellent thermal stability, as well as a mixture of the chitosan oligosaccharides prepared by the foregoing method.

According to the present invention, it is possible to provide a medicine and/or health supplement food which is effective in preventing and treating diabetes, including chitosan oligosaccharide that contains at least 40 wt. % of disaccharides without monosaccharide, and has excellent effects in terms of reducing HbAlc and blood glucose as well as excellent thermal stability. Therefore, the medicine and/or health supplement food including chitosan oligosaccharide according to the present invention may have superior effects in terms of reducing blood glucose, prevent and treat metabolic diseases as well as diabetes through reduction of HbAlc as a novel diagnosis item and a standard for assessment of long term blood glucose control, and prevent diabetes complications.

### BRIEF DESCRIPTION OF THE DRAWINGS

The above and other objects, features and other advantages of the present invention will be more clearly understood from the following detailed description taken in conjunction with the accompanying drawings, in which:

FIG. 1 is a graph showing thermal stabilities of chitosan oligosaccharide mixtures at 50° C. prepared in Examples 1 to 5 according to the present invention, in comparison with one another;

FIG. 2 is a graph showing a comparison of the results of the effects of inhibiting postprandial blood glucose rise in SD rats according to various chitosan oligosaccharides;

FIG. 3 is a graph showing a comparison of the results of the effects of inhibiting postprandial blood glucose rise in SD rats, according to the chitosan oligosaccharides prepared in Examples 1, 2 and 3, respectively.

FIG. 4 is a graph showing a comparison of the results of the effects of inhibiting postprandial blood glucose rise in SD rats, according to the chitosan oligosaccharides prepared in Examples 3, 4 and 5, respectively.

### DETAILED DESCRIPTION OF EMBODIMENTS

Hereinafter, the present invention will be described in more detail by the following examples. These examples of the present invention are proposed only to illustrate the present invention but, of course, do not restrict or limit the scope of the present invention to be protected. Subject matters easily conceived from a detailed description and examples of the present application by those skilled in the related art may be construed to be included in the scope of the present invention to be protected. Literature cited in the present disclosure is incorporated herein by reference.

### EXAMPLE

### Preparation of Chitosan Oligosaccharide

### Example 1

### Preparation of Chitosan Oligosaccharide Ascorbic Acid Salt

A chitosan oligosaccharide ascorbic acid salt was prepared according to a method in Example 1 disclosed in Korean Patent Registration No. 10-0506710, as described below.

First, after putting 55 g of chitosan with a degree of deacetylation of 99% into 500 ml of distilled water, the mixture was agitated for 10 minutes to prepare a chitosan suspension at a concentration of 11%. After adding 35 g of ascorbic acid to the chitosan suspension and agitating it for 2 hours to dissolve the same in the suspension, it was adjusted to pH 5.0 using ascorbic acid. 55 units of chitosanase (Sigma, C0794, chitosanase from Streptomyces sp.) were added to degrade chitosan at 38° C. for 12 hours. After heating the resultant product at 80° C. for 10 minutes, the enzyme was inactivated then filtered out and the remaining chitosan oligosaccharide composition was freeze dried at −40° C., thus resulting in 80 g of chitosan oligosaccharide ascorbic acid salt powder.

As a result of analyzing the obtained chitosan oligosaccharide ascorbic acid salt (powder) using an HPLC device (column: Asahipak NH2-50P 4E), glucosamine as a monosaccharide was not detected and the chitosan oligosaccharide had a degree of polymerization ranging from 2 to 10. Further, as a result of analyzing the same by an AOAC method (‘AOAC Official Method’) (hereinafter, referred to as “AOAC method”: Glucosamine in Raw Materials and Dietary Supplements Containing Glucosamine Sulfate and/or Glucosamine Hydrochloride High Performance Liquid Chromatography with FMOC-Su Derivatization, 2005, 01), which was partially modified, a content of oligoglucosamine was 54 wt. %.

A compositional ratio of constituents per saccharide of the obtained dried material was analyzed using a MALDI-TOF mass analyzer (Voyager DE, Perkin-Elmer Co.). According to analyzed results of the constitutional composition, the obtained product contained 15.6 wt. % of disaccharide, 17.3 wt. % of trisaccharide, 24.8 wt. % of tetrasaccharide, 21.2 wt. % of penta-saccharide, 9.8 wt. % of hexa-saccharide, 5.8 wt. % of hepta-saccharide, 3.7 wt. % of octa-saccharide, and 1.7 wt. % of other polysaccharides higher than octa-saccharide. Further, since a ratio of tetra- and penta-saccharides in a total content of oligosaccharide was 45 wt. % or more, both the tetra- and penta-saccharide were confirmed as major components. The chitosan oligosaccharide ascorbic acid salt prepared in Example 1 was named as ‘COS-C(H)’ and used in the following experiments for the assessment of effects thereof.

### Example 2

### Preparation of Chitosan Oligosaccharide Acetate Salt

A chitosan oligosaccharide acetate salt was prepared according to a method in Example 3 described in U.S. Pat. No. 5,981,510, as described below.

First, after putting 250 g of chitosan extracted from crab shells into 5 L of distilled water and adding 90 g of glacial acetic acid, the mixture was agitated overnight to prepare a viscous chitosan solution. After adding 50 mg of chitosanase derived from Bacillus pumilus (chitosanase BN-262, 47,000 unit/g powder, purchased from Bioprogen, Republic of Korea) to the chitosan solution, the mixture was placed in a thermal water bath at 40° C. to react therein for 18 hours. After completing the reaction, enzymes were inactivated by heating the reaction product at 80° C. for 10 minutes and a chitosan oligosaccharide solution was prepared. Then, spray drying was executed to yield 200 g of chitosan oligosaccharide acetate salt in dried powder form.

As a result of analyzing the obtained chitosan oligosaccharide acetate salt, glucosamine as a monosaccharide was not detected while a content of oligoglucosamine was 67 wt. % as measured by an AOAC method. A compositional ratio of constituents per saccharide of the obtained dried material was analyzed using a MALDI-TOF mass analyzer (Voyager DE, Perkin-Elmer Co.). According to analyzed results of the constitutional composition, the obtained product contained 21 wt. % of disaccharide, 27 wt. % of trisaccharide, 20 wt. % of tetrasaccharide, 14 wt. % of penta-saccharide, 10 wt. % of hexa-saccharide, 6 wt. % of hepta-saccharide, and 2 wt. % of octa-saccharide, which was substantially similar to the constitutional composition disclosed in the U.S. Pat. No. 5,981,510. The chitosan oligosaccharide acetate salt prepared in Example 2 was named as ‘COS-A’ and used in the following experiments for the assessment of effects thereof.

### Example 3

### Preparation of Chitosan Oligosaccharide Ascorbic Acid Salt Having Low Average Molecular Weight

According to the preparation methods described in Examples 1 and 2 above, the chitosan oligosaccharide was prepared by firstly dissolving chitosan in an organic acid to form a high-viscosity chitosan solution, and then, adding an enzyme to the high-viscosity chitosan solution, wherein a time for enzymatic degradation was 12 hours or more while having a large average molecular weight.

In Example 3, the enzyme was firstly added to chitosan to reduce the average molecular weight while further adding an organic acid to the chitosan slowly to dissolve the chitosan, so as to prepare chitosan oligosaccharide without viscous properties. Further, a reaction time was reduced.

100 g of chitosan was put into 2,000 ml of distilled water, and was swollen at 80° C. for 2 hours. After cooling the swollen chitosan to 40° C., 300 units of chitosanase derived from Bacillus Circulans (purchased from Kunpoong Bio Co. Ltd.) were added thereto. 65 g of ascorbic acid was added to a mixed solution of chitosan and enzyme at an interval of 10 minutes bit by bit over six additions. After adding the acid, the mixture was reacted at 40° C. for 8 hours. After completing the reaction, the reaction product was heated at 70° C. for 30 minutes to inactivate the enzyme, followed by spray drying, thus resulting in 140 g of chitosan oligosaccharide ascorbic acid salt in dried powder form.

A content of oligoglucosamine was 54 wt. % as measured by an AOAC method and, according to analyzed results of the constitutional composition by a MALDI-TOF mass analyzer, the obtained product contained 48.7 wt. % of disaccharide, 26.7 wt. % of trisaccharide, 14.1 wt. % of tetrasaccharide, 6.0 wt. % of penta-saccharide, 2.6 wt. % of hexa-saccharide and 1.9 wt. % of hepta-saccharide. Therefore, it was demonstrated that a ratio of disaccharide in a total content of oligosaccharide was 45 wt. % or more, thus being considerably high. The chitosan oligosaccharide ascorbic acid salt prepared in Example 3 was named as ‘COS-C(L)’ and used in the following experiments for the assessment of effects thereof.

### Example 4

### Preparation of Chitosan Oligosaccharide Succinic Acid Salt Having Low Average Molecular Weight

The chitosan oligosaccharide was prepared according to the same procedures as described in Example 3, and the organic acid used herein was prepared using 40 g of succinic acid instead of 65 g of ascorbic acid. A content of oligoglucosamine was 62 wt. % as measured by an AOAC method and, according to analyzed results of the constitutional composition by a MALDI-TOF mass analyzer, the obtained product contained 44.8 wt. % of disaccharide, 25.4 wt. % of trisaccharide, 17.7 wt. % of tetrasaccharide, 7.5 wt. % of penta-saccharide, 3.0 wt. % of hexa-saccharide and 1.6 wt. % of hepta-saccharide. Therefore, it was demonstrated that a ratio of disaccharide in a total content of oligosaccharide was about 45 wt. %, thus being considerably high. The chitosan oligosaccharide succinic acid salt prepared in Example 4 was named as ‘COS-S’ and used in the following experiments for the assessment of effects thereof.

### Example 5

### Preparation of Mixture of Chitosan Oligosaccharide Organic Acid Salts Having Low Average Molecular Weight

The chitosan oligosaccharide was prepared according to the same procedures as described in Example 3, and the organic acid used herein was a mixture of 70 wt. % of ascorbic acid and 40 wt. % succinic acid and 60 g of the mixture was used. A content of oligoglucosamine was 57 wt. % as measured by an AOAC method and, according to analyzed results of the constitutional composition by a MALDI-TOF mass analyzer, the obtained product contained 47.2 wt. % of disaccharide, 19.5 wt. % of trisaccharide, 17.2 wt. % of tetrasaccharide, 10.7 wt. % of penta-saccharide, 3.3 wt. % of hexa-saccharide and 2.1 wt. % of hepta-saccharide. Therefore, it was demonstrated that a ratio of disaccharide in a total content of oligosaccharide was 45 wt. % or more, thus being considerably high. The mixture of chitosan oligosaccharide ascorbic acid salt and chitosan oligosaccharide succinic acid salt prepared in Example 5 was named as ‘COS-C/S’ and used in the following experiments for the assessment of effects thereof.

(Thermal Stability Test)

### Example 6

### Thermal Stabilities of Individual Chitosan Oligosaccharide Salts

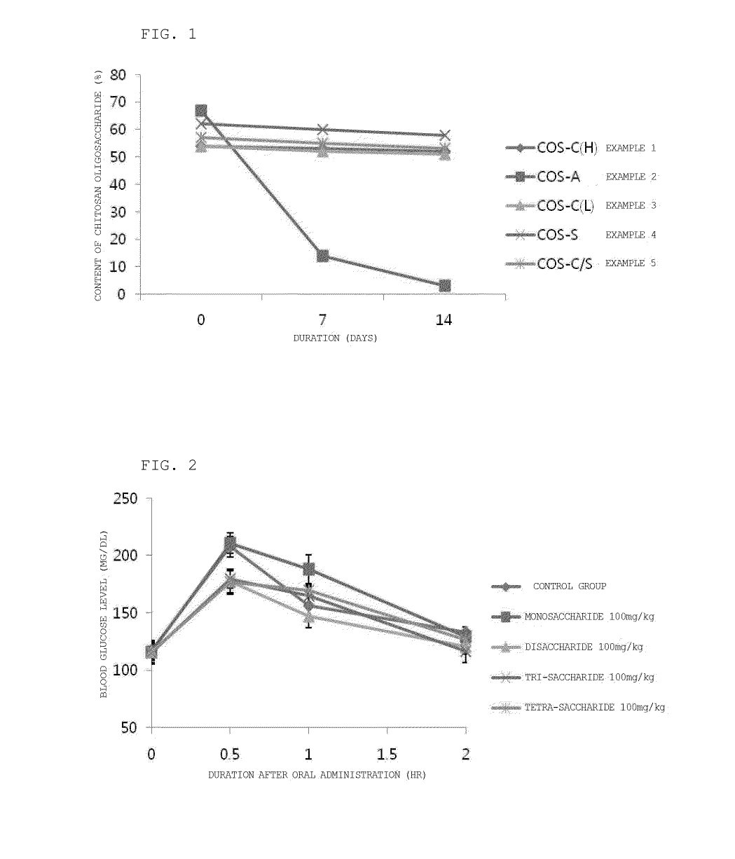

Thermal stability is an important factor to retain and assess quality of a product during product distribution. Therefore, in order to assess the thermal stability of each of the chitosan oligosaccharide mixtures prepared in Examples 1 to 5, a change in content of chitosan oligosaccharide of the prepared mixture under a condition of 50° C. was investigated and results thereof are shown in FIG. 1.

As illustrated in FIG. 1, when all of the chitosan oligosaccharide products were subjected to a preservation test at 50° C., the chitosan oligosaccharide ascorbic acid products (COS-C(H), COS-C(L)) were the most stable, while the chitosan oligosaccharide succinic acid product (COS-S) and the chitosan oligosaccharide ascorbic acid salt/chitosan oligosaccharide succinic acid salt product (COS-C/S) were relatively stable. Conversely, the chitosan oligosaccharide acetate salt product (COS-A) exhibited a decrease in content early and, since about day 14, it was detected that a content of chitosan oligosaccharide was not more than 5%, thus possibly causing a problem according to distribution environments. Therefore, the chitosan oligosaccharide acetate salt prepared according to the U.S. Pat. No. 5,981,510 as a prior art entailed a problem in terms of thermal stability, and may have a possibility that it causes a reduction of anti-diabetes effects due to a decrease of chitosan oligosaccharide during product distribution.

(Experiments for Effects of Chitosan Oligosaccharide)

### Experimental Example 1

### Effect of Inhibiting Blood Glucose Rise in Relation to Molecular Weight

Leaving the chitosan oligosaccharide products prepared in Examples 1 to 5 of the present disclosure aside, a glucose standard substance (purchased from Seikagaku, Japan) was used and the effects of inhibiting blood glucose rise after a meal (‘postprandial blood glucose rise’) per saccharide were measured. To SD rats, 100 mg/kg (based on body weight) of each of standard substances from monosaccharide to tetra-saccharide and 2 g/kg (based on body weight) of sucrose were orally administered by 1 ml/rat using an oral administration sonde. At 30 minutes, 60 minutes and 120 minutes after the oral administration, blood was collected from the tail vein of each SD rat and a change in blood glucose concentration in venous blood was measured using a blood glucose meter (CareSens II). With regard to sucrose, assessed results of the inhibitory efficacy of postprandial blood glucose rise per saccharide in SD rats are shown in FIG. 2.

As illustrated in FIG. 2, the inhibitory efficacy of postprandial blood glucose rise of chitosan oligosaccharide in relation to sucrose was assessed and results of the assessment demonstrated that a control group showed an increase in blood glucose of 207.8 mg/dl at 30 minutes after the meal, while three chitosan oligosaccharides, that is, the disaccharide, trisaccharide and tetra-saccharide were 177.6 mg/dl, 179.4 mg/dl and 176.5 mg/dl, respectively, thus showing the effects of inhibiting blood glucose rise by the foregoing three chitosan oligosaccharides. In particular, the disaccharide and trisaccharide had favorable effects in terms of inhibiting blood glucose rise even after 30 minutes, that is, a time to typically reach maximum blood glucose after a meal. Generally, it was demonstrated that the disaccharide having a smaller molecular weight in the chitosan oligosaccharide may have superior inhibitory efficacy in terms of blood glucose rise, as compared to tri- or tetra-saccharide having a relatively large molecular weight. However, monosaccharide was 210.7 mg/dl at 30 minutes after a meal, which is higher than 207.8 mg/dl in the control group, thus increasing blood glucose. Consequently, it was confirmed that chitin or chitosan oligosaccharide containing monosaccharide may have a problem in terms of blood glucose control. Accordingly, it can be understood that it is important to prepare a chitosan oligosaccharide composition without monosaccharide and rather with a higher content of disaccharide.

### Experimental Example 2

### Glucose Tolerance Effect in Relation to Preparation Method

The effects of inhibiting postprandial blood glucose rise by chitosan oligosaccharides prepared according to the methods described in Examples 1, 2 and 3, respectively, were measured. To SD rats, 0.5 g/kg (based on body weight) of each of the chitosan oligosaccharides COS-C(H), COS-A and COS-C(L) prepared according to the methods described in Examples 1, 2 and 3, respectively, as well as 2 g/kg of sucrose (based on body weight), were orally administered by 1 ml/rat using an oral administration sonde. At 30 minutes, 60 minutes and 120 minutes after the oral administration, blood was collected from the tail vein of each SD rat and a change in blood glucose concentration in venous blood was measured using a blood glucose meter (CareSens II).

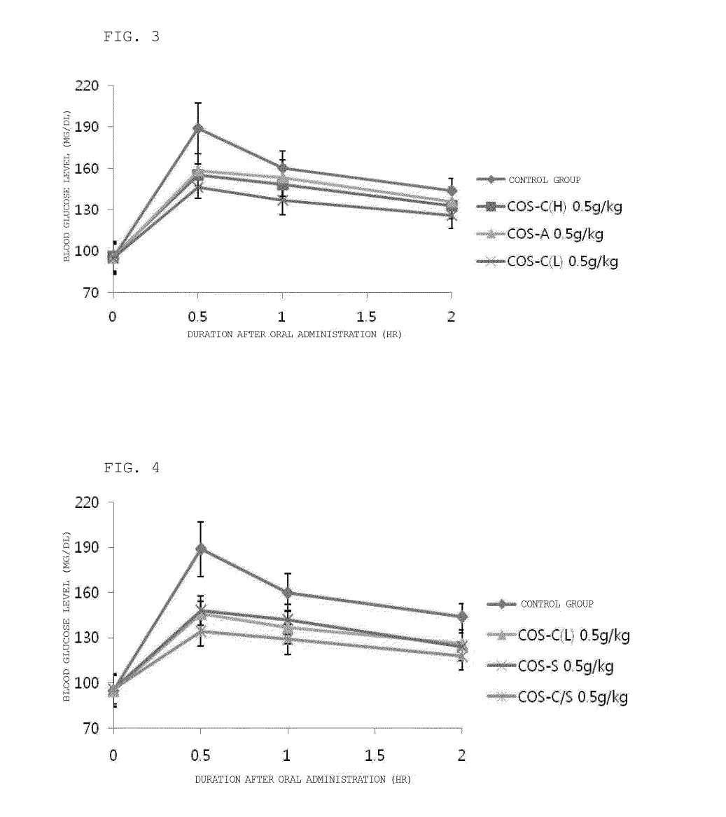

With regard to sucrose, assessed results of the inhibitory efficacies of postprandial blood glucose rise of chitosan oligosaccharides COS-C(H), COS-A and COS-C(L) according to Examples 1, 2 and 3 in SD rats are shown in FIG. 3.

As illustrated in FIG. 3, the inhibitory efficacies of postprandial blood glucose rise of chitosan oligosaccharides COS-C(H), COS-A and COS-C(L) prepared in Examples 1, 2 and 3, respectively, in relation to sucrose were assessed and results of the assessment demonstrated that a control group showed an increased blood glucose of 188.7 mg/dl at 30 minutes after the meal, while COS-C(H), COS-A and COS-C(L) were 155.1 mg/dl, 158.2 mg/dl and 145.8 mg/dl, respectively, thus showing the effects of inhibiting blood glucose rise by the foregoing three chitosan oligosaccharides. In particular, the chitosan oligosaccharide COS-C(L) having a higher content of disaccharide prepared by the method described in Example 3 exhibits superior effects in terms of inhibiting blood glucose, as compared to the chitosan oligosaccharides COS-C(H) and COS-A prepared by the methods described in Examples 1 and 2, respectively. Such a fact that COS-C(L) having a relatively high content of disaccharide has excellent inhibitory effects in terms of blood glucose rise suggests that a distribution of saccharides may greatly influence the inhibitory effects of blood glucose rise, in addition to the result demonstrated in Experimental Example 1 wherein chitosan oligosaccharide having disaccharide has excellent inhibitory effects in terms of blood glucose rise. In particular, it was demonstrated that the chitosan oligosaccharide mixture having a high content of disaccharide exhibited remarkably superior effects in terms of inhibiting blood glucose rise. Consequently, it can be understood that it is important to prepare a chitosan oligosaccharide composition having a high content of disaccharide without monosaccharide.

### Experimental Example 3

### Synergetic Glucose Tolerance Effect in Relation to Preparation Method

The effects of inhibiting postprandial blood glucose rise by each of the chitosan oligosaccharides presented according to the methods described in Examples 3, 4 and 5, respectively, were measured. The chitosan oligosaccharide mixtures (COS-C(L), COS-S and COS-C/S) used in the present experimental example are samples having characteristics of a high disaccharide distribution and relatively high thermal stability. To SD rats, 0.5 g/kg (based on body weight) of each of the chitosan oligosaccharide mixtures COS-C(L), COS-S and COS-C/S prepared according to the methods described in Examples 3, 4 and 5, respectively, as well as 2 g/kg of sucrose (based on body weight), were orally administered by 1 ml/rat using an oral administration sonde. At 30 minutes, 60 minutes and 120 minutes after the oral administration, blood was collected from the tail vein of each SD rat and a change in blood glucose concentration in venous blood was measured using a blood glucose meter (CareSens II).

With regard to sucrose, assessed results of the inhibitory efficacies of postprandial blood glucose rise of the chitosan oligosaccharide mixtures COS-C(L), COS-S and COS-C/S according to Examples 3, 4 and 5, respectively, in SD rats are shown in FIG. 4.

As illustrated in FIG. 4, the inhibitory efficacies of postprandial blood glucose rise of the chitosan oligosaccharide mixtures COS-C(L), COS-S and COS-C/S prepared in Examples 3, 4 and 5, respectively, in relation to sucrose were assessed and results of the assessment demonstrated that a control group showed an increased blood glucose of 188.7 mg/dl at 30 minutes after the meal, while COS-C(L), COS-S and COS-C/S were 145.8 mg/dl, 148.3 mg/dl and 134.3 mg/dl, respectively, thus showing the effects of inhibiting blood glucose rise by the foregoing three chitosan oligosaccharides. In particular, the chitosan oligosaccharide mixture COS-C/S having a higher content of disaccharide and containing chitosan oligosaccharide ascorbic acid salt and chitosan oligosaccharide succinic acid salt mixed in a ratio (by weight) of 7:3 prepared by the method described in Example 5 exhibits superior effects in terms of inhibiting blood glucose, as compared to the chitosan oligosaccharides COS-C(L) and COS-S prepared by the methods described in Examples 3 and 4, respectively. From these results, it can be understood that inhibitory effects in terms of blood glucose rise are different depending upon types of salts of chitosan oligosaccharide, and such inhibitory effects in terms of blood glucose rise may be improved by selection and combination of proper salts as well as a distribution of saccharides. In the following experimental examples, experiments have been executed to determine blood glucose reduction effects when administering a sample having excellent inhibitory effects in terms of postprandial blood glucose rise in relation to sucrose to an animal with disease for a long period of time.

### Experimental Example 4

### Experiments of Blood Glucose Descent Efficacy and Risk Factors of Diabetes Using a Disease Animal (db/db Mouse)

The chitosan oligosaccharide used in the present experimental example is the one having most excellent glucose tolerance effects in relation to sucrose, and was prepared by the method described in Example 5, and named ‘COS-C/S.’ As the disease animal, db/db mice were purchased (from the Central Lab. Animal Inc.) and used in the experiments. These were divided into three groups, that is, a control group, COS-C/S administration groups 1 and 2, and each group included 10 mice. The COS-C/S group 1 was subjected to administration of chitosan oligosaccharide beginning at week 3 after first dietary intake, that is, at a point of time that test animals showed a very high blood glucose. On the other hand, the COS-C/S group 2 was subjected to administration of chitosan oligosaccharide at the beginning of dietary intake with respect to a whole dietary intake period of time of 6 weeks (see Table 1 below).

TABLE 1
High carbohydrate dietary composition in disease animal
(db/db mouse)

| | Control group | COS-C/S 1, 2 |
|---|---|---|
| Corn starch | 661.0 | 621.0 |
| Casein | 226.0 | 226.0 |
| Soybean oil | 60.0 | 60.0 |
| Vitamin mixture | 31.0 | 31.0 |
| Mineral mixture | 9.0 | 9.0 |
| Calcium phosphate | 10.0 | 10.0 |
| Sodium chloride | 3.0 | 3.0 |
| Test sample | 0 | 40 |

COS-C/S group 1: administration of chitosan oligosaccharide beginning at week 3 after first dietary intake
COS-C/S group 2: administration of chitosan oligosaccharide at the beginning of dietary intake

Meanwhile, an HbAlc value standardized and used in association with blood glucose is adopted as an important test item to predict a danger of diabetes complications and determine as to whether a long term blood glucose control is required. For reference, according to recommendations of the United States Diabetes Society, average blood glucose levels were estimated for 2 to 3 months and standardized into numerical values in terms of percentage (%) of HbAlc, which have been used as a diagnosis standard of diabetes since 2010.

TABLE 2
Correlation between eAG (Estimated
Average Glucose) and HbAlc value

| HbAlc (%) | eAG (mg/dL) | eAG (mmol/L) |
|---|---|---|
| 5.0 | 97 | 5.4 |
| 5.5 | 111 | 6.2 |
| 6.0 | 126 | 7.0 |
| 6.5 | 140 | 7.8 |
| 7.0 | 154 | 8.6 |
| 7.5 | 169 | 9.4 |
| 8.0 | 183 | 10.2 |
| 8.5 | 197 | 11.0 |
| 9.0 | 212 | 11.8 |
| 9.5 | 226 | 12.6 |
| 10.0 | 240 | 13.4 |
| 10.5 | 255 | 14.2 |
| 11.0 | 269 | 14.9 |
| 11.5 | 283 | 15.7 |
| 12.0 | 298 | 16.5 |

eAG: Estimated Average Glucose
eAG (mg/ml) = 28.7 × HbAlc (%) − 46.7
eAG (mmol/ml) = 1.59 × HbAlc (%) − 2.59

After completing the dietary intake for 6 weeks, in order to measure a change in fasting blood glucose and a change in an HbAlc value in the disease animal, after fasting a test animal for 24 hours or more before dissection, blood was collected from the tail vein and a change in blood glucose concentration in venous blood was measured using a blood glucose meter (CareSens II) while a change in an HbAlc value was measured using a HbAlc meter (Easy A1C, Infopia Co., Ltd.). After completing the dietary intake of the disease animal (db/db mouse) over 6 weeks, a change in fasting blood glucose and a change in an HbAlc value were measured and such measured results are shown in Table 3 below.

TABLE 3
Fasting blood glucose and HbAlc value after dietary
intake of disease animal (db/db mouse) for 6 weeks

| | Blood glucose level | |
|---|---|---|
| | mg/dl | HbAlc % |
| Control group | 489.1 ± 65 | 10.58 ± 1.11 |
| COS-C/S group 1 | 272.5 ± 17** | 7.50 ± 1.25** |
| COS-C/S group 2 | 162.9 ± 37*** | 5.80 ± 0.80*** |

COS-C/S group 1: administration of chitosan oligosaccharide beginning at week 3 after first dietary intake
COS-C/S group 2: administration of chitosan oligosaccharide at the beginning of dietary intake

With regard to the fasting blood glucose after 6 weeks-dietary intake in the experiment using a disease animal (db/db mouse), the control group had 489.1 mg/dl, COS-C/S group 1 (with administration of chitosan oligosaccharide beginning at week 3 after first dietary intake) had 272.5 mg/dl, and COS-C/S group 2 (with administration of chitosan oligosaccharide at the beginning of dietary intake) had 162.9 mg/dl, respectively. Such results demonstrated that COS-C/S group 1 has a blood glucose value of 56% while COS-C/S group 2 has a blood glucose value of 33%, as compared to the control group, thus exhibiting the effects of remarkably reducing blood glucose by the intake of chitosan oligosaccharide.

For HbAlc, COS-C/S group 1 and COS-C/S group 2 had a level of about 71% and 55%, respectively, as compared to the control group, thus exhibiting a remarkable decrease in an HbAlc value by the intake of chitosan oligosaccharide.

The diabetes disease animal, that is, db/db mouse generally begin to suffer from diabetes around an age of 10 weeks and has a fasting blood glucose reaching about 400 mg/dl. However, when the animal was treated with chitosan oligosaccharide from the beginning of dietary intake, it became to have almost normal blood glucose. On the other hand, when starting the treatment of the animal with chitosan oligosaccharide at week 3 after first dietary intake, the blood glucose tended to be somewhat reduced. These results suggest that an intake of chitosan oligosaccharide may be helpful for the prevention and treatment of diabetes.

Further, when the HbAlc value exceeds 10%, it is diagnosed that a risk for diabetes complications is very high. Since the chitosan oligosaccharide administration groups also exhibited a decrease in an HbAlc value, it is understood that a long term intake of chitosan oligosaccharide may assure significant effects in treatment of diabetes and be helpful for prevention of diabetes complications.

### Experimental Example 5

### Clinical Trials of Chitosan Oligosaccharide

Although many functional products have been advertised or informed to the public on the basis of results provided by animal experiments, many materials do not exhibit desired effects in clinical trials, unlike the results of animal experiments. Further, according to published articles, the probability that results of animal experiments are indicative of the results of clinical trials was only 8.3% and only 0.3% of the foregoing cases reported that the results of the related animal experiments coincide with those of the clinical trials. Therefore, alternative clinical trials independent of animal experiments are still significantly needed (Andrew Knight, Proc. 6th World Congress on Alternatives & Animal Use in the Life Sciences, August 21 to 25, 2007).

A reason of such a fact as described above is to be why verification of efficacies through clinical trials in human beings is different from that in animal models, and effective ingredients react or are active in the human body in different ways from that in animals. In order to investigate such problems, clinical trials were executed to identify or verify efficacies with regard to the effects of reducing HbAlc and blood glucose by chitosan oligosaccharides.

The clinical trials were conducted with 60 participants from Yonsei University with IRB approval. An average age of the participants was 54.40±2.02 years for a test group and 57.88±1.79 years for a control group, an average height was 165.32±0.34 cm for the test group and 164.42±1.31 cm for the control group, and an average body weight was 66.68±1.79 kg for the test group and 64.49±1.96 kg for the control group. Therefore, there was no significant difference in initial values between both the foregoing groups (p>0.05). In terms of body mass index (BMI), body fat amount, percentage by body fat amount, systolic and diastolic blood pressures, or the like, there was no substantial difference in initial values between both the foregoing groups.

For the test group, chitosan oligosaccharide having a content of oligoglucosamine of 54% was taken with water via oral administration three times every day (500 mg/time) before meals for 12 weeks, while roasted barley flour as a control substance was orally administered to the control group.

For the dietary control, all participants were instructed before the beginning of human experimentations to have the same meals as usual and exercise throughout a period of experimentation, and intake calories and spent calories were set for individual participants. For assessment, food intake amounts during 0 week, 6 weeks, 12 weeks, and 3 days before a visit (2 days among weekdays and 1 day in a weekend) were recorded.

According to random assignment, participants suitable to standards for selection of subjects were assigned to both the groups. Blood glucose testing was performed by collecting blood at 0 minutes during a fast after fasting for 12 hours, and then, collecting blood from a vein at 30 minutes, 60 minutes and 120 minutes after the participant drank a solution of 75 g glucose in 250 to 350 ml water over 5 to 15 minutes, and measuring blood glucose using an oxidation enzyme method. HbAlc was determined by collecting venous blood after fasting for 12 hours, and measuring HbAlc through turbidimetry using an immunoturbidimetric analyzer.

TABLE 4
Results of clinical trials for glucose/HbA1c

| | | | | | |
|---|---|---|---|---|---|
| Glucose | Placebo | | Test | | |
| (mg/dl) | (n = 26) | P^{a} | (n = 25) | P^{a} | P^{b} |

| | | | | | |
|---|---|---|---|---|---|
| 0 minute | | | | | |
| Before | 118.58 ± 3.23 | 0.637 | 115.36 ± 3.18 | 0.927 | |
| After | 119.42 ± 3.15 | | 114.12 ± 2.56 | | |
| Change | 0.85 ± 2.27 | | −1.24 ± 1.86 | | 0.692 |
| 30 minutes | | | | | |
| Before | 197.81 ± 8.80 | 0.170 | 196.52 ± 7.15 | 0.013 | |
| After | 183.88 ± 6.96 | | 178.56 ± 6.90 | | |
| Change | −13.92 ± 8.08 | | −17.96 ± 6.92 | | 0.534 |
| 60 minutes | | | | | |
| Before | 214.87 ± 13.89 | 0.316 | 222.00 ± 10.79 | 0.028 | |
| After | 221.96 ± 12.31 | | 202.72 ± 12.50 | | |
| Change | 7.15 ± 9.62 | | −19.28 ± 8.22 | | 0.030 |
| 120 minutes | | | | | |
| Before | 172.65 ± 11.79 | 0.629 | 165.96 ± 12.33 | 0.028 | |
| After | 175.27 ± 10.97 | | 167.88 ± 13.99 | | |
| Change | 2.62 ± 8.29 | | 1.92 ± 11.08 | | 0.888 |
| Glu_AUC | | | | | |
| Before | 376.02 ± 19.49 | 0.751 | 376.60 ± 16.92 | 0.061 | |
| After | 375.93 ± 16.48 | | 353.82 ± 18.21 | | |
| Change | −0.08 ± 13.13 | | −22.79 ± 12.30 | | 0.200 |
| HbA1c (%) | | | | | |
| Before | 6.19 ± 0.12 | 0.238 | 6.36 ± 0.19 | 0.023 | |
| After | 6.28 ± 0.13 | | 6.11 ± 0.11 | | |
| Change | 0.09 ± 0.06 | | −0.26 ± 0.15 | | 0.021 |

Means ± S.E.
p^{a}—values derived from paired t-test with Wilcoxon signed rank test
P^{b}—values derived from independent t-test with Mann-Whitney U-test

After adult men and women suffering from impaired glucose tolerance had the intake of chitosan oligosaccharide or placebo over 12 weeks, blood indexes as well as fasting blood glucoses were analyzed. For the test group (25 persons), the blood glucose at 30 minutes was initially 196.52±7.15 mg/dL but significantly reduced to 178.56±6.90 mg/dL after 12 weeks, while the blood glucose at 60 minutes was initially 222.00±10.79 mg/dL but significantly reduced to 202.72±12.50 mg/dL (see Table 4).

Further, for the test group, an area under the curve (AUC) for showing blood glucose response was initially 376.60±16.92 mg/dL but decreased to 353.82±18.21 mg/dL after 12 weeks. However, the control group (26 persons) did not exhibit a significant change. Comparing a change in blood glucose index between groups, the blood glucose at 60 minutes of the test group was reduced by 19.28±8.22 mg/dL to an initial value to thus have a significant difference from the change shown in the control group. According to analyzed results of HbAlc as a diabetes-related indicator, the test group had initial HbAlc of 6.36±0.19% but HbAlc after 12 weeks of 6.11±0.11%, which was significantly reduced (see Table 4). Comparing a change in HbAlc between groups, the test group had a decrease in HbAlc of 0.26±0.15% to the initial value, thus exhibiting a significant difference from the change shown in the control group.

From the foregoing results, it is understood that a chitosan oligosaccharide mixture is not toxic, and may reduce blood glucose of a person with glucose tolerance and also significantly decrease an HbAlc value as a blood glucose control factor through the human experiment, thereby preventing and treating diabetes and being possibly applied to medicines and/or health supplement foods for prevention of diabetes complications.

Hereinabove, preferred embodiments have been described to more concretely understand the present invention. However, it will be apparent to those skilled in the art that the present invention is not particularly restricted to such embodiments but various modifications and alterations thereof may be possible without departing from the scope and spirit of the present invention, and such modifications and alterations are duly included in the present invention as defined by the appended claims.

What is claimed is:

1. A method for preparation of a chitosan oligosaccharide composition, comprising:
adding chitosan to distilled water to swell the chitosan;
firstly treating the chitosan with a chitosanase to prevent the chitosan solution from becoming a state of being viscose, then, adding an organic acid to a mixed solution of the chitosan and chitosanase at a predetermined time interval bit by bit over several additions;
allowing mutual reaction between the chitosan, chitosanase and organic acid; and
inactivating the chitosanase to obtain the chitosan oligosaccharide composition.

2. The method according to claim 1, wherein the obtained chitosan oligosaccharide composition contains at least 40 wt. % of disaccharide in chitosan oligosaccharide to 100 wt. % of the composition without monosaccharide.

3. The method according to claim 2, wherein the obtained chitosan oligosaccharide composition has the effects of reducing a glycosylated hemoglobin (HbAlc) value and blood glucose.

4. The method according to claim 3, wherein the composition is formulated at an administration dose of 300 to 3,000 mg daily in terms of oligoglucosamine content, in order to have the effects of reducing an HbAlc value and blood glucose.

5. The method according to claim 1, wherein the organic acid is added to the mixed solution of chitosan and chitosanase at an interval of 10 minutes bit by bit over six additions.

6. The method according to claim 1, wherein the organic acid is at least one selected from a group consisting of ascorbic acid and succinic acid.

7. The method according to claim 1, wherein the obtained chitosan oligosaccharide is a chitosan oligosaccharide ascorbic acid salt, chitosan oligosaccharide succinic acid salt or a mixture thereof.

8. A food composition for prevention and treatment of diabetes, comprising a mixture of chitosan oligosaccharide ascorbic acid salt and chitosan oligosaccharide succinic acid salt.

9. The composition according to claim 8, wherein a content of the chitosan oligosaccharide succinic acid salt ranges from 5 to 35 wt. % to 100 wt. % of the mixture.

10. The composition according to claim 8, wherein a ratio by weight of the chitosan oligosaccharide ascorbic acid salt to chitosan oligosaccharide succinic acid salt is 7:3.

11. The composition according to claim 8, wherein the composition is in a food form of any one of drinks, beverages, tea bags, capsules, powder, and pressed tablets.

12. A pharmaceutical composition for prevention and treatment of diabetes, comprising a mixture of chitosan oligosaccharide ascorbic acid salt and chitosan oligosaccharide succinic acid salt.

13. The composition according to claim 12, wherein a content of the chitosan oligosaccharide succinic acid salt ranges from 5 to 35 wt. % to 100 wt. % of the mixture.

14. The composition according to claim 12, wherein a ratio by weight of the chitosan oligosaccharide ascorbic acid salt to chitosan oligosaccharide succinic acid salt is 7:3.

15. The composition according to claim 12, wherein the composition is manufactured into a formulation for oral administration of any one of tablets, pills, hard/soft capsules, liquids, suspensions, emulsions, syrups, granules, and elixirs.
